(19) United States
(12) Patent Application Publication
(10) Pub. No.: US 2020/0061054 A1
(43) Pub. Date: Feb. 27, 2020

(54) USE OF DIANHYDROGALACTITOL OR ANALOGS AND DERIVATIVES IN COMBINATION WITH A P53 MODULATOR OR A PARP INHIBITOR

(71) Applicant: Del Mar Pharmaceuticals (BC) Ltd., Vancouver (CA)

(72) Inventors: Jeffrey A. Bacha, Vancouver (CA); Dennis M. Brown, Menlo Park, CA (US); Anne Steinø, Vancouver (CA); Mads Daugaard, Vancouver (CA); Beibei Zhai, Vancouver (CA)

(73) Assignee: Del Mar Pharmaceuticals (BC) Ltd., Vancouver, BC (CA)

(21) Appl. No.: 16/489,122

(22) PCT Filed: Feb. 28, 2018
(86) PCT No.: PCT/US18/20314

§ 371 Date: Aug. 27, 2019

Related U.S. Application Data
(60) Provisional application No. 62/464,763, filed on Feb. 28, 2017.

(51) Int. Cl. A61K 31/496; A61K 31/4436; A61K 31/336; A61K 31/7048; A61P 35/00; A61K 31/047

(52) U.S. Cl. CPC A61K 31/496; A61K 31/4436; A61K 31/047; A61K 31/7048; A61P 35/00; A61K 31/336

(57) ABSTRACT

Methods and compositions employing dianhydrogalactitol or a derivative analog of dianhydrogalactitol together with a p53 modulator, a PARP inhibitor, or topoisomerase inhibitor for treatment of malignancies are provided.

### CROSS-REFERENCE(S) TO RELATED APPLICATION(S)

This application claims the benefit of U.S. Provisional Application No. 62/464,763 filed Feb. 28, 2017, the disclosure of which is expressly incorporated herein by reference in its entirety.

### BACKGROUND OF THE INVENTION

Despite recent advances in cancer treatment methods, cancer continues to be one of the most common and deadly diseases. For difficult to treat cancers, a patient's treatment options are often exhausted quickly resulting in a desperate need for additional treatment regimens. Even for the most limited of patient populations, any additional treatment opportunities would be of considerable value.

Particularly treatment-resistant malignancies include, for example, non-small cell lung cancer (particularly cisplatin-resistant non-small cell lung carcinoma NSCLC); prostate cancer; O6-methylguanine-DNA methyltransferase (MGMT)-unmethylated pediatric high-grade glioma (HGG); Sonic hedgehog (SHH) or group 3 medulloblastoma, which may be p53-mutated; platinum-resistant ovarian high-grade serous ovarian cancer (HGSOC) (high-grade serous ovarian cancer); which may have BRCA1/2 mutation or BRCAness; cisplatin-resistant bladder cancer; platinum-resistant triple-negative breast cancer; and triple-negative breast cancer with metastases to the brain.

There is a continuing need for compositions and methods to treat cancer.

### SUMMARY OF THE INVENTION

The present invention is generally directed to methods and compositions employing an alkylating hexitol derivative, such as dianhydrogalactitol or a derivative or analog of dianhydrogalactitol, together with a topoisomerase inhibitor, a PARP inhibitor such as olaparib, or a p53 modulator such as nutlin-3, for treatment of a malignancy. Typically, the malignancy is: non-small cell lung cancer (particularly cisplatin-resistant NSCLC); prostate cancer; MGMT-unmethylated pediatric high-grade glioma (HGG); SHH or group 3 medulloblastoma, which may be p53-mutated; platinum-resistant ovarian high-grade serous ovarian cancer (HGSOC) (high-grade serous ovarian cancer); which may have BRCA1/2 mutation or BRCAness; cisplatin-resistant bladder cancer; platinum-resistant triple-negative breast cancer; or triple-negative breast cancer with metastases to the brain.

In one aspect, disclosed herein is a composition for treating a malignancy comprising an alkylating hexitol, a p53 modulator, and optionally one or more pharmaceutically acceptable carriers.

In certain embodiments, the p53 modulator is nutlin-3a or GSK2830371. In other embodiments, the alkylating hexitol derivative is dianhydrogalactitol, diacetyldianhydrogalactitol, or dibromodulcitol. In still other embodiments, the alkylating hexitol derivative is dianhydrogalactitol.

In some embodiments, both the alkylating hexitol and the p53 modulator are included in a therapeutically effective quantity, for example, in quantities that produce synergism between the activities of the alkylating hexitol derivative and the p53 modulator. The alkylating hexitol derivative and the p53 modulator can be included in separate containers, each of which can optionally comprise a pharmaceutically acceptable carrier. In some embodiments, the composition comprises a treatment regimen.

In another aspect, provided herein is a method for treating a malignancy comprising the steps of:

(a) administering a therapeutically effective quantity of an alkylating hexitol derivative; and

(b) administering a therapeutically effective quantity of a p 53 modulator;

in order to treat the malignancy.

Preferably, the alkylating hexitol derivative is dianhydrogalactitol, diacetyldianhydrogalactitol, or dibromodulcitol. In some embodiments, the alkylating hexitol derivative is dianhydrogalactitol. In other embodiments, the p53 modulator is nutlin-3a or GSK2830371.

In certain embodiments, the malignancy to be treated by the methods disclosed herein is an ovarian cancer, including platinum-resistant ovarian cancer, is BRCA-deficient ovarian cancer, BRCAness ovarian cancer, a high-grade serous ovarian carcinoma, and BRCA-proficient ovarian cancer.

The steps of the methods described above can be performed in any particular order, including sequentially in any order or simultaneously.

In yet another aspect, disclosed herein is a composition for treating a malignancy comprising a therapeutically effective quantity of an alkylating hexitol, a therapeutically effective quantity of a PARP inhibitor, and optionally one or more pharmaceutically acceptable carriers.

Preferably, the alkylating hexitol derivative is dianhydrogalactitol, diacetyldianhydrogalactitol, or dibromodulcitol. In some embodiments, the alkylating hexitol derivative is dianhydrogalactitol. In other embodiments, the PARP inhibitor is olaparib, talazoparib, niraparib, or rucaparib. In some embodiments, the composition comprises dianhydrogalactitol and olaparib.

In some embodiments, both the alkylating hexitol derivative and the PARP inhibitor are included in a therapeutically effective quantity, for example, in quantities that produce synergism between the activities of the alkylating hexitol derivative and the PARP inhibitor. The alkylating hexitol derivative and the PARP inhibitor can be included in separate containers, each of which can optionally comprise a pharmaceutically acceptable carrier. In some embodiments, the composition comprises a treatment regimen.

In yet another aspect, disclosed herein is a method for treating a malignancy comprising the steps of:

(a) administering a therapeutically effective quantity of an alkylating hexitol derivative; and

(b) administering a therapeutically effective quantity of a PARP inhibitor; in order to treat the malignancy.

In certain embodiments, the malignancy to be treated by the methods disclosed herein is an ovarian cancer, including platinum-resistant ovarian cancer, is BRCA-deficient ovarian cancer, BRCAness ovarian cancer, a high-grade serous ovarian carcinoma, and BRCA-proficient ovarian cancer.

The steps of the methods described above can be performed in any particular order, including sequentially in any order or simultaneously.

In yet another aspect, disclosed herein is a method for treating a malignancy comprising the steps of:

(a) administering a therapeutically effective quantity of an alkylating hexitol derivative; and

(b) administering a therapeutically effective quantity of a topoisomerase inhibitor;

in order to treat the malignancy.

Preferably, the alkylating hexitol derivative is dianhydrogalactitol, diacetyldianhydrogalactitol, or dibromodulcitol. In some embodiments, the alkylating hexitol derivative is dianhydrogalactitol. In other embodiments, the topoisomerase inhibitor is a Type 1 topoisomerase inhibitor, Type 2 topoisomerase inhibitor, or a Type 1/Type 2 topoisomerase inhibitor. Preferably, the topoisomerase inhibitor is camptothecin, irinotecan, doxorubicin, topotecan, etoposide, or mitoxantrone.

In certain embodiments, the malignancy to be treated by the methods disclosed herein is non-small cell lung cancer (NSCLC), including is cisplatin-resistant NSCLC. In other embodiments, the malignancy is prostate cancer.

The steps of the methods described above can be performed in any particular order, including sequentially in any order or simultaneously.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 shows VAL-083 cytotoxicity against BRCA1-proficient (BRCA1+/+) and -deficient (BRCA1−/−) ovarian cancer cells A2780. The data shows that dianhydrogalactitol possesses increased activity in ovarian cancers with impaired BRCA, such as approximately 20% of ovarian cancers. The potency of dianhydrogalactitol activity was increased (IC₅₀ was reduced) when the homologous recombination (HR) pathway was impaired by BRCA knockdown, further demonstrating that dianhydrogalactitol-induced DNA-lesions are repaired via the HR pathway.

FIG. 2 shows that dianhydrogalactitol exhibits super-additivity in combination with an exemplary PARP inhibitor olaparib in ovarian cancer cell line A2780 (n=3).

FIGS. 3A and B show results of a pilot study investigating several non-DNA targeting agents (BKM120, GSK2830371, MK-1775, Nutlin-3a, and palbociclib) alone or in combination with 0.15 uM (A) or 0.3 uM (B) VAL-083 against ovarian cancer cell line A2780 cells, which have wild-type p53. The data demonstrates that p53 activator nutlin-3a augmented the activity of dianhydrogalactitol.

FIGS. 4A-4D demonstrate that VAL-083 (DAG) and GSK2830371 show synergy in A2780 cell line at the indicated concentrations.

FIGS. 5A-5D demonstrate that VAL-083 (DAG) and nutlin-3a show synergy in A2780 cell line at the indicated concentrations.

FIGS. 6A and 6B show that combination of VAL-083 (DAG) with Nutlin-3a or GSK2830371 induces synergistic upregulation of p53-dependent response in ovarian cancer cell line A2780.

### DETAILED DESCRIPTION OF THE INVENTION

Disclosed herein are methods and compositions employing an alkylating hexitol derivative such as dianhydrogalactitol or a derivative or analog of dianhydrogalactitol together with a PARP inhibitors or p53 modulator such as nutlin-3a that can be used for treatment of a malignancy. Additionally, as described below, the present invention also encompasses methods and compositions employing an alkylating hexitol derivative together with topoisomerase inhibitors.

Typically, the malignancy is non-small cell lung cancer (particularly cisplatin-resistant NSCLC); prostate cancer; or platinum-resistant ovarian high-grade serous ovarian cancer (HGSOC); which may have BRCA1/2 mutation or BRCAness, meaning deficiencies in the homologous recombination (HR) DNA repair pathway other than BRCA1/2, leading to impaired HR. The methods and compositions disclosed herein can be employed as either first-line or second-line therapy.

### Dianhydrogalactitol and Derivatives and Analogs of Dianhydrogalactitol

Alkylating hexitol derivatives that can be used in compositions and methods according to the present invention include galactitols, substituted galactitols, dulcitols, and substituted dulcitols. Typically, the alkylating hexitol derivative is selected from the group consisting of dianhydrogalactitol, derivatives of dianhydrogalactitol, analogs of dianhydrogalactitol, diacetyldianhydrogalactitol, derivatives of diacetyldianhydrogalactitol, analogs of diacetyldianhydrogalactitol, dibromodulcitol, derivatives of dibromodulcitol, and analogs of dibromodulcitol. More typically, the alkylating hexitol derivative is selected from the group consisting of dianhydrogalactitol, derivatives of dianhydrogalactitol, diacetyldianhydrogalactitol, derivatives of diacetyldianhydrogalactitol, dibromodulcitol, and derivatives of dibromodulcitol. In some embodiments, the alkylating hexitol derivative is dianhydrogalactitol.

As used herein, the terms “substituted hexitol derivative” or “alkylating hexitol derivative” encompass these alternatives unless specifically limited to a compound, a compound with defined substituents, or a class of compounds within the broad definitions provided above.

In some embodiments, the alkylating hexitol is dianhydrogalactitol, including its stereoisomers. The terms “dianhydrogalactitol,” “DAG,” and “VAL-083” are used herein interchangeably. The structure of dianhydrogalactitol (DAG or VAL-083) is shown in Formula (I), below.

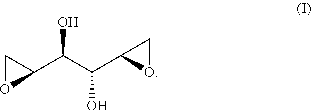

The galactitols, substituted galacitols, dulcitols, and substituted dulcitols included in the methods and combinations disclosed herein are either alkylating agents or prodrugs of alkylating agents, as discussed further below. Also within the scope of the invention are derivatives of dianhydrogalactitol that, for example, have one or both hydrogens of the two hydroxyl groups of dianhydrogalactitol replaced with lower alkyl, have one or more of the hydrogens attached to the two epoxide rings replaced with lower alkyl, or have the methyl groups present in dianhydrogalactitol and that are attached to the same carbons that bear the hydroxyl groups replaced with C2-C6 lower alkyl or substituted with, for example, halo groups by replacing a hydrogen of the methyl group with, for example a halo group. As used herein, the term “halo group,” without further limitation, refers to one of fluoro, chloro, bromo, or iodo. As used herein, the term “lower alkyl,” without further limitation, refers to C1-C6 groups and includes methyl. The term “lower alkyl” can be further limited, such as “C2-C6 lower alkyl,” which excludes methyl. The term “lower alkyl”, unless further limited, refers to both straight-chain and branched alkyl groups. These groups can, optionally, be further substituted, for example, with halo groups.

In some embodiments, the alkylating hexitol derivative is diacetyldianhydrogalactitol. The structure of diacetyldianhydrogalactitol is shown in Formula (II), below.

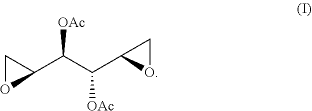 [SMILES: CC(=O)O[C@H]([C@H](OC(C)=O)[C@@H]1CO1)[C@@H]1CO1]

Also within the scope of the invention are derivatives of diacetyldianhydrogalactitol that, for example, have one or both of the methyl groups that are part of the acetyl moieties replaced with C2-C6 lower alkyl, have one or both of the hydrogens attached to the epoxide ring replaced with lower alkyl, or have the methyl groups attached to the same carbons that bear the acetyl groups replaced with lower alkyl or substituted with, for example, halo groups by replacing a hydrogen with, for example, a halo group.

In other embodiments, the alkylating hexitol derivative is dibromodulcitol of Formula (III):

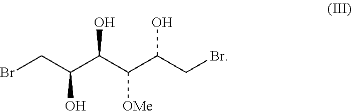 [SMILES: CO[C@H]([C@H](O)[C@@H](O)CBr)[C@H](O)CBr]

Dibromodulcitol can be produced by the reaction of dulcitol with hydrobromic acid at elevated temperatures, followed by crystallization of the dibromodulcitol. Some of the properties of dibromodulcitol are described in N. E. Mischler et al., “Dibromoducitol,” Cancer Treat. Rev. 6: 191-204 (1979). In particular, dibromodulcitol, as an a, w-dibrominated hexitol, dibromodulcitol shares many of the biochemical and biological properties of similar drugs such as dibromomannitol and mannitol myleran. Activation of dibromodulcitol to the diepoxide dianhydrogalactitol occurs in vivo, and dianhydrogalactitol may represent a major active form of the drug; this means that dibromogalactitol has many of the properties of a prodrug. Absorption of dibromodulcitol by the oral route is rapid and fairly complete. Dibromodulcitol has known activity in melanoma, breast lymphoma (both Hodgkins and non-Hodgkins), colorectal cancer, acute lymphoblastic leukemia and has been shown to lower the incidence of central nervous system leukemia, non-small cell lung cancer, cervical carcinoma, bladder carcinoma, and metastatic hemangiopericytoma.

Also within the scope of the invention are derivatives of dibromodulcitol that, for example, have one or more hydrogens of the hydroxyl groups replaced with lower alkyl, or have one or both of the bromo groups replaced with another halo group such as chloro, fluoro, or iodo.

The compounds described herein may contain one or more chiral centers and therefore, may exist as stereoisomers, such as enantiomers or diastereomers. The invention includes each of the isolated stereoisomeric forms (such as the enantiomerically pure isomers and other alternatives for stereoisomers) as well as mixtures of stereoisomers in varying degrees of chiral purity or percentage, including racemic mixtures and mixtures of diastereomers unless a specific stereoisomer is specified. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. When the chemical name does not specify the isomeric form of the compound, it denotes any one of the possible isomeric forms or mixtures of those isomeric forms of the compound.

The compounds may also exist in several tautomeric forms, and the depiction herein of one tautomer is for convenience only, and is also understood to encompass other tautomers of the form shown. Accordingly, the chemical structures depicted herein encompass all possible tautomeric forms of the illustrated compounds.

As used herein, the term “solvate” means a compound formed by solvation (the combination of solvent molecules with molecules or ions of the solute), or an aggregate that consists of a solute ion or molecule, i.e., a compound of the invention, with one or more solvent molecules. When water is the solvent, the corresponding solvate is “hydrate.” Examples of hydrate include, but are not limited to, hemihydrate, monohydrate, dihydrate, trihydrate, hexahydrate, and other water-containing species. It should be understood by one of ordinary skill in the art that the pharmaceutically acceptable salt, and/or prodrug of the present compound may also exist in a solvate form. The solvate is typically formed via hydration which is either part of the preparation of the present compound or through natural absorption of moisture by the anhydrous compound of the present invention.

Additional derivatives of dianhydrogalactitol are known in the art. These derivatives include dimethyldianhydrogalactitol and disuccinyldianhydrogalactitol and are disclosed in Y. Zhou et al., “Research Progress in New Anti-Cancer Drugs with Hexitols,” Chin. J. Cancer 12: 257-260 (1993).

In some alternatives, the derivative or analog of dianhydrogalactitol can be a prodrug. As used herein, the term “prodrug” refers to compounds that are transformed in vivo to yield a disclosed compound or a pharmaceutically acceptable form of the compound. In some embodiments, a prodrug is a compound that may be converted under physiological conditions or by solvolysis to a biologically active compound as described herein. Thus, the term “prodrug” refers to a precursor of a biologically active compound that is pharmaceutically acceptable. A prodrug can be inactive when administered to a subject, but is then converted in vivo to an active compound, for example, by hydrolysis (e.g., hydrolysis in blood or a tissue). In certain cases, a prodrug has improved physical and/or delivery properties over a parent compound from which the prodrug has been derived. The term “prodrug” is also meant to include any covalently bonded carriers which release the active compound in vivo when the prodrug is administered to a subject. Prodrugs of a therapeutically active compound, as described herein, can be prepared by modifying one or more functional groups present in the therapeutically active compound in such a way that the modifications are cleaved, either in routine manipulation or in vivo, to yield the parent therapeutically active compound.

When multiple therapeutic agents are administered according to the methods disclosed herein, each therapeutic agent can be administered separately, or two or more therapeutic agents can be administered in a single pharmaceutical composition. For example, when three therapeutic agents are to be administered, the following possibilities exist. (1) Each of the three therapeutic agents is administered individually; in this case, each agent can be administered in a separate pharmaceutical composition or as the agent alone without use of a pharmaceutical composition for the agent. (2) Two of the therapeutic agents are administered together in a single pharmaceutical composition, while the third therapeutic agent is administered separately, either as the agent alone or in a separate pharmaceutical composition. (3) All three therapeutic agents are administered together in a single pharmaceutical composition.

### Poly (ADP-Ribose) Polymerase (PARP) Inhibitors

In certain embodiments, the compositions and methods disclosed herein include a combination of an alkylating hexitol derivative and a PARP inhibitor. Inhibitors of the enzyme poly-ADP ribose polymerase (PARP) have been developed for multiple indications, especially for treatment of malignancies. Several forms of cancer are more dependent on the activity of PARP than are non-malignant cells.

The enzyme PARP catalyzes the polymerization of poly-ADP ribose chains, typically attached to a single-strand break in cellular DNA. The coenzyme NAD+ is required as a substrate for generating ADP-ribose monomers to be polymerized; nicotinamide is the leaving group during polymerization, in contrast to pyrophosphate which is the leaving group during normal DNA or RNA synthesis, which leaves a pyrophosphate as the linking group between adjacent ribose sugars in the chain rather than phosphate as occurs in normal DNA or RNA. The PARP enzyme comprises four domains: a DNA-binding domain, a caspase-cleaved domain, an auto-modification domain, and a catalytic domain. The DNA-binding domain comprises two zinc finger motifs. In the presence of damaged DNA, the DNA-binding domain will bind the DNA and induce a conformational shift. PARP can be inactivated by caspase-3 cleavage, which is a step that occurs in programmed cell death (apoptosis).

Several PARP enzymes are known, including PARP1 and PARP2. Of these two enzymes, PARP1 is responsible for most cellular PARP activity. The binding of PARP1 to single-strand breaks in DNA through the amino-terminal zinc finger motifs recruits XRCC1, DNA ligase III, DNA polymerase β, and a kinase to the nick. This is known as base excision repair (BER). PARP2 has been shown to oligomerize with PARP1, and the oligomerization stimulates catalytic activity. PARP2 is also therefore implicated in BER.

PARP1 inhibitors display their cytotoxic effect through two mechanisms: 1) by inhibiting the activity of PARP1 and thus inhibiting the repair of single-strand breaks in DNA, and 2) by trapping PARP on the DNA strand also preventing repair of the single strand break. Both of these mechanisms lead to strand breaks. When such breaks are unrepaired, DNA double-strand breaks during the subsequent DNA replication can induce double-strand breaks. The proteins BRCA1 and BRCA2 among others are involved in the repair of double-strand breaks in DNA by the error-free homologous recombination (HR) repair pathway. In tumors with mutations in the genes BRCA1, BRCA2 or other genes involved in HR, these double-strand breaks cannot be efficiently repaired, leading to cell death. Normal cells do not replicate their DNA as frequently as tumor cells, and normal cells are generally HR-proficient and can thus repair these double-strand breaks through homologous recombination (HR) repair. Therefore, normal cells are less sensitive to the activity of PARP inhibitors than tumor cells which have BRCA1/2 deficiencies or BRCAness.

Some tumor cells that lack the tumor suppressor PTEN may be sensitive to PARP inhibitors because of downregulation of Rad51 (BRCAness), a critical homologous recombination (HR) component. Tumor cells that are low in oxygen are also sensitive to PARP inhibitors as HR is down-regulated in cells undergoing hypoxic stress.

PARP inhibitors are also considered potential treatments for other life-threatening diseases, including stroke and myocardial infarction, as well as for long-term neurodegenerative diseases (G. Graziani & C. Szabo, “Clinical Perspectives of PARP Inhibitors,” Pharmacol. Res. 52: 109-118 (2005)).

A number of PARP inhibitors are known in the art. PARP inhibitors include, but are not limited to, niraparib, iniparib, talazoparib, olaparib, rucaparib, veliparib, CEP-9722 (a prodrug of CEP-8983 (11-methoxy-4,5,6,7-tetrahydro-1H-cyclopenta[a]pyrrolo[3,4-c]carbazole-1,3(2H)-dione), MK 4827 ((S)-2-(4-(piperidin-3-yl)phenyl)-2H-indazole-7-carboxamide), and BGB-290, all of which are intended to be within the scope of the present invention and specified formulas.

In some embodiments, the PARP inhibitor is olaparib. Olaparib is a PARP inhibitor, inhibiting poly-ADP ribose polymerase (PARP), an enzyme involved in DNA repair, and trapping PARP onto the DNA strand. It can be effective in treating cancers with BRCA1 or BRCA2 mutations or with genetic or epigenetic alterations leading to phenotypes similar to BRCA1/2 mutated (termed BRCAness). As used herein, “BRCAness” means a deficiency in the homologous recombination DNA repair pathway other than BRCA leading to phenotypes similar to those with BRCA1/2 mutated. Inhibition and trapping of PARP block the repair of spontaneously occurring DNA lesions, in particularly single-stranded DNA nicks of which about 10,000 occur per day in each cell, which then turn into much more severe DNA double-strand breaks (DSBs). BRCA1/2 mutations and BRCAness alterations block or reduce the effectiveness of other DNA repair pathways, specifically the homologous recombination (HR) pathway involved in DSB repair, leading to cancer cell death.

In other embodiments, other PARP inhibitors can be useful for inclusion the compositions and methods of their use disclosed herein, for example, olaparib, talazoparib, niraparib and rucaparib.

Platinum-based chemotherapy is the standard of care in treating ovarian cancer. However, p53 mutations, as exhibited by 96% of HGSOC cases, often confer resistance to platinum-based drugs. As it has been previously shown, VAL-083 has been tested against panel of ovarian cancer cell lines and was active in all cell lines tested: (a) wild type p53 (A2780), (b) knocked out (A2780 p53−/−), and (c) mutant p53 (2780CP, OVCAR10, HEY and OVCA-433) harboring P72R, V172F, and/or G266R mutations. VAL-083 circumvents cisplatin-resistance in multiple ovarian cancer models, including HGSOC cell-lines, independently of p53 mutations and has demonstrated clinical activity against ovarian cancer. In addition, VAL-083 activity is independent of other DNA repair pathways implicated in resistance to cis-platin and PARP inhibitors, and the cancer cells thus rely heavily on the HR pathway for repair of VAL-083-induced DNA double strand breaks. Combined, these data support VAL-083 for the treatment of Pt-resistant ovarian cancer, particularly ovarian cancer with deficient homologous recombination repair (HR). Therefore, a combination therapy comprising VAL-083 and PARPi can be effective for the treatment of platinum-resistant ovarian cancers, including HGSOC. VAL-083 can synergize with PARP inhibitors, maximizing DNA double strand breaks and inhibiting DNA repair, thus overwhelming DNA repair capacity and directing ovarian cancer cells to apoptotic cell death in cisplatin-resistant and HR-dysfunctional ovarian cancers.

### Other Agents

In some embodiments, the compositions disclosed herein comprise an alkylating hexitol derivative and another agent, such as an agent that does not target DNA, for example, a p53 modulator. As used herein, a p53 modulator is an agent that modulates the activity of p53, for example, without directly interacting with p53.

In some embodiments, such non-DNA targeting agent or p53 modulator is nutlin-3a, also known as (−)-Nutlin-3, (−)-4-(4,5-Bis(4-chlorophenyl)-2-(2-isopropoxy-4-methoxyphenyl)-4,5-dihydro-1H-imidazole-1-carbonyl)piperazin-2-one, 4-[[(4S,5R)-4,5-Bis(4-chlorophenyl)-2-(2-isopropoxy-4-methoxyphenyl)-4,5-dihydroimidazol-1-yl]carbonyl]piperazin-2-one, and 4-[[(4S,5R)-4,5-Bis(4-chlorophenyl)-4,5-dihydro-2-[4-methoxy-2-(1-methylethoxy)phenyl]-1H-imidazol-1-yl]carbonyl]-2-Piperazinone. Nutlin-3a is a cis-imidazoline analog that inhibits the interaction between MDM2 and the tumor suppressor p53, and thereby stabilizes p53 and inhibits tumor growth (L. T. Vassilev et al., “In Vivo Activation of the p53 Pathway by Small-Molecule Antagonists of MDM2,” Science 303: 844-848 (2004)).

In other embodiments, the compositions and methods described herein comprise an alkylating hexitol derivative and GSK2830371. GSK2830371 or (S)-5-(((5-Chloro-2-methylpyridin-3-yl)amino)methyl)-N-(3-cyclopentyl-1-(cyclopropylamino)-1-oxopropan-2-yl)thiophene-2-carboxamide or 5-[[(5-Chloro-2-methyl-3-pyridinyl)amino]methyl]-N-[(1S)-1-(cyclopentylmethyl)-2-(cyclopropylamino)-2-oxoethyl]-2-thiophenecarboxamide is an orally active, allosteric inhibitor of wild-type p53-induced phosphatase (Wip1, also known as PPM1D/PP2Cδ), an oncogenic type 2C serine/threonine phosphatase that negatively regulates key proteins in the DNA damage-response pathway. It binds to a flap subdomain that regulates Wip1's enzymatic activity and substrate recognition.

In certain embodiments, the compositions comprise a therapeutically effective quantity of dianhydrogalactitol and a therapeutically effective quantity of nutlin-3a. In other embodiments, the compositions comprise a therapeutically effective quantity of dianhydrogalactitol and a therapeutically effective quantity of GSK2830371. In certain embodiments, the therapeutically effective quantities are the quantities that produce synergism between the activities of the alkylating hexitol derivative and the additional agent, such as nutlin-3a or GSK2830371.

### Topoisomerase Inhibitors

In certain embodiments, the compositions and methods disclosed herein include a combination of an alkylating hexitol derivative and a topoisomerase inhibitor. Topoisomerase inhibitors useful for inclusion in the compositions and methods disclosed herein include inhibitors of Type 1 topoisomerase and inhibitors of Type 2 topoisomerase. As alkylating hexitol derivatives, such as VAL-083, induce cell cycle arrest in S- followed by G2/M-phase, agents that require cancer cells to be in S/G2-phase for maximum effect, including topoisomerase inhibitors, can have synergistic effect with VAL-083.

Inhibitors of Type 1 topoisomerase include, but are not limited to, irinotecan, topotecan, camptothecin, homocamptothecin, DB 67 (7-t-butyldimethylsilyl-10-hydroxy-camptothecin), lamellarin D, indotecan, indimitecan, karenitecan, exatecan, lurtotecan, gimatecan, and belotecan, and others known in the art.

Inhibitors of Type 2 topoisomerase include two main classes: (i) topoisomerase poisons, which target the topoisomerase-DNA complex, and (ii) topoisomerase inhibitors, which disrupt catalytic turnover. Topoisomerase poisons that target eukaryotic topoisomerases include, but are not limited to, actinomycin D, daunomycin, amsacrine, etoposide, etoposide phosphate, teniposide, and doxorubicin. Topoisomerase inhibitors, which target the N-terminal ATPase domain of Type 2 topoisomerase and inhibit the turnover of the enzyme, include, but are not limited to, ICRF-193 (4-[2-(3,5-dioxo-1-piperazinyl)-1-methylpropyl]piperazine-2,6-dione) and genistein. Other inhibitors of Type 2 topoisomerase include, but are not limited to, amonafide and derivatives and analogs thereof, mitoxantrone, ellipticines, and aurintricarboxylic acid. Still other inhibitors of both Type 1 and Type 2 topoisomerase known in the art can be included in the compositions and methods disclosed herein.

### Methods and Compositions for Treatment of a Malignancy

In one aspect, disclosed herein is a method for treating a malignancy comprising the steps of:

(1) administering a therapeutically effective quantity of an alkylating hexitol derivative; and

(2) administering a therapeutically effective quantity of a p53 modulator;

in order to treat the malignancy.

In some embodiments, the p53 modulator is Nutlin-3a. In other embodiments, the p53 modulator is GSK2830371. In certain embodiments, the therapeutically effective quantities are the quantities that produce synergism and/or super-additivity between the activities of the alkylating hexitol derivative and the additional agent, such as nutlin-3a or GSK2830371.

In certain embodiments, the malignancy is ovarian cancer. In some embodiments, the cancer is BRCA-proficient, BRCA-deficient, or BRCAness cancer. Ovarian cancers that can be treated by the methods disclosed herein include cancers that have resistance to platinum-based therapies, such as cis-platin, and cancers that have impaired homologous recombination repair mechanism.

Yet another aspect relates to a method for treating a malignancy comprising the steps of:

(1) administering a therapeutically effective quantity of an alkylating hexitol derivative; and

(2) administering a therapeutically effective quantity of topoisomerase inhibitor;

in order to treat the malignancy.

In another aspect, disclosed herein is a method for treating a malignancy comprising the steps of:

(1) administering a therapeutically effective quantity of an alkylating hexitol derivative; and

(2) administering a therapeutically effective quantity of a PARP inhibitor;

in order to treat the malignancy.

In the aspects of the methods described above, typically, the alkylating hexitol derivative is selected from the group consisting of dianhydrogalactitol, a derivative, analog, or prodrug of dianhydrogalactitol, diacetyldianhydrogalactitol, a derivative, analog, or prodrug of diacetyldianhydrogalactitol, dibromodulcitol, and a derivative, analog, or prodrug of dibromodulcitol. Preferably, the alkylating hexitol derivative is dianhydrogalactitol or a derivative, analog, or prodrug of dianhydrogalactitol. More preferably, the alkylating hexitol derivative is dianhydrogalactitol. In another alternative, the alkylating hexitol derivative is diacetyldianhydrogalactitol or a derivative, analog, or prodrug of diacetyldianhydrogalactitol. Preferably, in this alternative, the alkylating hexitol derivative is diacetyldianhydrogalactitol.

Each of the steps (a) and (b) of the methods disclosed herein can include administration of a single dose of the agent or a series of doses. For example, the step of administering a therapeutically effective quantity of an alkylating hexitol derivative (step (a)) includes administration of a series of doses administered over a certain time period during a treatment cycle, for example, 40 mg/m2/day×3 days every 21 days.

It is understood that the steps (a) and (b) of the methods described herein can be performed in any particular order. In certain embodiments, the steps (a) and (b) can be performed simultaneously, for example, the therapeutically effective quantities of the alkylating hexitol derivative and the additional agent, such as p53 modulator, PARP inhibitor, or topoisomerase inhibitor are co-administered. In other embodiments, the steps are performed sequentially, for example the alkylating hexitol derivative can be administered before or after the administration of the additional agent such as p53 modulator, PARP inhibitor, or topoisomerase inhibitor, i.e., step (a) is performed before step (b) or step (b) is performed prior to step (a).

Yet another aspect of the present invention relates to compositions for treating a malignancy comprising:

(a) a therapeutically effective quantity of an alkylating hexitol derivative selected from the group consisting of dianhydrogalactitol, a derivative, analog, or prodrug of dianhydrogalactitol, diacetyldianhydrogalactitol, and a derivative, analog, or prodrug of diacetyldianhydrogalactitol;

(b) a therapeutically effective quantity of an additional agent, wherein the additional agent is a PARP inhibitor, nutlin-3a, or GSK2830371; and

(c) optionally, a pharmaceutically acceptable carrier.

As used herein, a “composition” can comprise one or more agents, such as DAG and a p53 modulator, one or more PARP inhibitors, and/or one or more topoisomerase inhibitors. The composition can comprise each of the agents combined in a single container with a pharmaceutically acceptable carrier, or the composition can comprise each of the active agents in a separate container with a pharmaceutically acceptable carrier, which can be either the same or different; wherein the composition comprises a treatment regimen.

In some embodiments, the therapeutically effective quantities are the quantities of the alkylating hexitol derivative and the additional agent such as PARP inhibitor, nutlin-3a, or GSK2830371, that produce synergism between the activities of the alkylating hexitol derivative and the additional agent.

Typically, in these compositions, the alkylating hexitol derivative is dianhydrogalactitol (DAG or VAL-083).

The methods and compositions disclosed herein can be employed as either first-line or second-line therapy or can be used as adjunct therapy or in combination with another method of cancer treatment.

The amount of a given pharmacologically active agent, such as an alkylating hexitol derivative such as dianhydrogalactitol or an analog or derivative of dianhydrogalactitol as described above, a topoisomerase inhibitor, or a PARP inhibitor, or a p53 modulator, including, but not limited to, olaparib, GSK2830371, or nutlin-3a, that is included in a unit dose of a pharmaceutical composition according to the present invention will vary depending upon factors such as the particular compound, disease condition and its severity, the identity (e.g., weight) of the subject in need of treatment, but can nevertheless be routinely determined by one skilled in the art.

Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. In some embodiments, the alkylating hexitol derivative and an additional agent, such as a PARP inhibitor, nutlin-3a, or GSK283037, are included in the composition separate pharmaceutical carriers. In other embodiments, the alkylating hexitol derivative and an additional agent, such as a PARP inhibitor, nutlin-3a, or GSK283037, are included in a single pharmaceutical carrier.

A pharmacologically active agent, as the agents of the methods and compositions described above, can be administered in conventional dosage form prepared by combining a therapeutically effective amount of such a pharmacologically active agent as an active ingredient with appropriate pharmaceutical carriers or diluents according to conventional procedures. These procedures may involve mixing, granulating and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like. In the case of the use of a pharmaceutical composition according to the present invention to treat glioma by inhibiting glycolysis, the dose can be optimized for a particular patient by monitoring the output of a product of glycolysis for the patient.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the particular complex being used, the particular composition formulated, the mode of administration and the particular site, host and disease and/or condition being treated. Actual dosage levels of the active ingredients in the pharmaceutical compositions of the present invention can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20th ed., 2000. Optimal dosages for a given set of conditions can be ascertained by those skilled in the art using conventional dosage-determination tests in view of the experimental data for an agent. For oral administration, an exemplary daily dose generally employed is from about 0.001 to about 3000 mg/kg of body weight, with courses of treatment repeated at appropriate intervals. In some embodiments, the daily dose is from about 1 to 3000 mg/kg of body weight. Other dosages are as described above.

Typical doses in a patient for the alkylating hexitol derivative may be anywhere between about 500 mg to about 3000 mg, given once or twice daily, e.g., 3000 mg can be given twice daily for a total dose of 6000 mg. In one embodiment, the dose is between about 1000 to about 3000 mg. In another embodiment, the dose is between about 1500 to about 2800 mg. In other embodiments, the dose is between about 2000 to about 3000 mg. Typically, doses are from about 1 mg/m²/day to about 50 mg/m²/day. Preferably, doses are from about 5 mg/m²/day to about 40 mg/m²/day. Additional alternatives for dosages are as described above with respect to schedules of administration and dose modification. When dosing is performed on a cycle, such as, but not limited to, a 21-day to a 33-day cycle, absolute quantities of total dosage depend on the number of dosing days per cycle and duration of the cycle. Dosages can be varied according to therapeutic response.

In some embodiments, when dianhydrogalactitol and an additional anti-neoplastic agent of the methods and compositions disclosed herein, e.g., a topoisomerase inhibitor, are administered together to treat a malignancy, dianhydrogalactitol is administered at doses of about 40 mg/m²/day×3 days every 21 days. In other embodiments, dianhydrogalactitol is dosed at about 60, about 67.5, or at about 75 mg/m²/day once weekly. In yet other embodiments, for example, when administered according to the methods described herein with a topoisomerase or a PARP inhibitor, dianhydrogalactitol is administered at doses lower than about 40 mg/m²/day.

Topoisomerase inhibitors, PARP inhibitors, GSK2830371, or nutlin-3a can be administered at the same time or close together in time as an alkylating hexitol derivative. The dosages of the dianhydrogalactitol and the other agent can be selected to provide a synergistic or superadditive effect. The dose can be further optimized as disclosed above.

Plasma concentrations in the subjects for the alkylating hexitol derivative can be between about 100 ng/mL to about 1200 ng/mL. In some embodiments, the plasma concentration can be between about 200 ng/mL to about 1000 ng/mL. In other embodiments, the concentration is about 300 ng/mL to about 600 ng/mL. In still other embodiments the plasma concentration is between about 600 ng/ml to about 1000 ng/mL. Administration of prodrugs is typically dosed at weight levels, which are chemically equivalent to the weight levels of the fully active form. These plasma concentrations can be further optimized to achieve the desired therapeutic effect.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

The compounds may be formulated for parenteral administration by injection, e.g., by bolus injection or continuous infusion. Formulations for injection may be presented in unit-dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Pharmaceutical compositions according to the present invention are usually administered to the subjects on multiple occasions. Intervals between single dosages can be weekly, monthly or yearly. Intervals can also be irregular as indicated by therapeutic response or other parameters well known in the art. Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the subject can be administered a prophylactic regime.

For the purposes of the present application, treatment can be monitored by observing one or more of the improving symptoms associated with the disease, disorder, or condition being treated, or by observing one or more of the improving clinical parameters associated with the disease, disorder, or condition being treated. As used herein, the terms “treatment,” “treating,” or equivalent terminology are not intended to imply a permanent cure for the disease, disorder, or condition being treated. Compositions and methods according to the present invention are not limited to treatment of humans, but are applicable to treatment of socially or economically important animals, such as dogs, cats, horses, cows, sheep, goats, pigs, and other animal species of social or economic importance. Unless specifically stated, compositions and methods according to the present invention are not limited to the treatment of humans.

The invention is illustrated by the following Examples which are included for illustrative purposes only, and are not intended to limit the invention.

### Examples

Materials and Methods

For Western blotting, cells were lysed in EBC buffer (50 mM Tris-HCl, pH 8.0, 120 mM NaCl, 1% NP-40, and 1 mM EDTA) supplemented with phosphatase inhibitor and protease inhibitor. Cellular proteins were separated by SDS-PAGE and transferred onto PVDF membrane. After incubation with blocking buffer for 1 h, the membranes were incubated with designated primary antibodies overnight at 4° C. Then, Membranes were washed three times for 10 min and incubated with horseradish peroxidase-conjugated anti-mouse or anti-rabbit antibodies for 1-2 h. Membranes were washed with TBST three times and developed with ECL system (Pierce) according to the manufacturer's instruction. The following primary antibodies were used for immunoblotting: γH2A.X (Cell Signaling Technology, 2577); H2A.X (Abcam, ab11175); phospho-ATM (S1981) (Rockland Antibodies and Assays, 200-301-400); ATM (Cell Signaling Technology, 2873); GAPDH (Cell Signaling Technology, 5174); phospho-RPA32 (S33) (Bethyl Laboratories, A300-246A); phospho-CHK1 (S345) (Cell Signalling Technology, 2348), phospho-CHK2 (T68) (Cell Signaling Technology, 2661), Cyclin A2 (Abcam, ab16726), RPA32 (Abcam, ab2175).

For crystal violet assay, following 72 h of different concentrations of VAL-083 treatment, cells were fixed in 1% glutaraldehyde for 5 min. After rinsing with distilled water, cells were incubated with 0.1% crystal violet solution dye for 10 min. Cells were then gently washed with distilled water and air-dried. The crystals on the plate were dissolved in Sorenson's solution before reading absorbance at 560 nm wavelength in a microplate reader. Cell growth is expressed as percentage compared to untreated cells.

For cell cycle analysis using propidium iodide (PI) staining, cell cycle distribution was evaluated based on DNA content using PI staining. Serum starvation synchronized cells were treated with 5 μM VAL-083 for 1 h, 4 h, 19 h, 24 h, 44 h, and 49 h. Cells were then trypsinized, washed in PBS, and centrifuged at 1000 rpm for 5 min. Cell pellets were fixed in 70% ethanol overnight at 4° C. After washing, cells were incubated with 500 μl, PI solution in PBS containing 50 μg/ml PI, 100 μg/mL RNase A, and 0.05% Triton X-100 for 40 min at 37° C. in the dark. Thereafter, cells were washed and re-suspended in PBS. DNA content was analyzed by flow cytometry and histograms were made using FlowJo software. Untreated cells were included as control.

For immunofluorescence (IF), cells were grown on glass coverslips for at least 16 h before serum starvation for 24 h. Synchronized cells were treated with VAL-083 for 1 h followed by washout and incubation with complete medium for another 24 h. Subsequently, cells were fixed for 30 min with 4% paraformaldehyde in PBS at room temperature. Then, cells were washed three times with PBS and permeabilized for 20 min with 0.5% Triton X-100 in PBS. After washing with PBS for three times and blocking with 3% BSA in PBS for 1 h at room temperature, cells were incubated overnight at 4° C. with primary antibodies diluted in fresh blocking solution. Next, cells were washed three times with PBS and incubated with secondary antibodies for 1 h at room temperature. After washing with PBS for three times, coverslips were mounted with Vectashield mounting medium (with DAPI). Antibodies used in IF staining were γH2A.X (Cell Signaling Technology, 2577); Cyclin A2 (Abcam, ab16726); donkey anti-rabbit Alexa-Fluor 594 (Life Technologies, A21207) and donkey anti-mouse Alexa-Fluor 488 (Life Technologies, A21202). Images were acquired using a Zeiss AxioObserver.

Combination of Dianhydrogalactitol (VAL-083) and PARP Inhibitors in A2780 Cells with or without BRCA1-Kd

Knockdown (kd) of BRCA1 in ovarian cancer cell line A2780 to induce defective homologous recombination (HR) was accomplished by using a siRNA protocol (G. He et al., “The Impact of S- and G2-Checkpoint Response on the Fidelity of G1-Arrest by Cisplatin and Its Comparison to a Non-Cross-Resistant Platinum(IV) Analog,” Gynecol. Oncol. 122: 402-409 (2011); G. He et al., “Recruitment of Trimeric Proliferating Cell Nuclear Antigen by G1-Phase Cyclin-Dependent Kinases Following DNA Damage with Platinum-Based Antitumour Agents,” Br. J. Cancer 109: 2378-2388 (2013); X. Xie et al., “Heterozygous p53(V172F) Mutation in Cisplatin-Resistant Human Tumor Cells Promotes MDM4 Recruitment and Decreases Stability and Transactivity of p53,” Oncogene 35: 4798-4806 (2016)). A concentration of 40 nM siRNA for 24 hr was highly effective in reducing BRCA1 in two independent experiments. Cytotoxicity was determined by MTT assay using the standard 5-day drug exposure protocol.

Ovarian A2780 tumor cells were treated with control or BRCA1 siRNA in 6-well plates and 24 hr later were washed, typsinized and aliquoted to 96-well plates. After a further 24 hr to allow cell attachment, cells were exposed to VAL-083 and/or one of the PARP inhibitors at concentrations that individually give in control cells about a 20-25% fractional affect (Fa or growth inhibition). After 5 days, the cells are processed for the MTT assay.

Based on inspection of Fa data, BRCA1 knockdown (kd) increased cellular sensitivity to the agents of interest, as follows:
VAL-083: 1.4- to 1.7-fold
Olaparib: 6- to 8-fold
Talazoparib: 1.5- to 2.0-fold
Niraparib: 2.0- to 3.0-fold
Rucaparib: ˜3.0-fold
Veliparib: 2.0- to 3.0-fold

In control cells (no BRCA1 kd), combination of VAL-083 with each PARP inhibitor showed 2-24% increase in activity above that predicted for simple additivity in each case. Specific data are as follows:
VAL-083+Olaparib: 18.4%
VAL-083+Talazoparib: 12.5%
VAL-083+Niraparib: 24.0%
VAL-083+Rucaparib: 6.2%
VAL-083+Veliparib: 2.0%

In BRCA1-siRNA treated cells (˜90% BRCA1 kd), combination of VAL-083 with each PARPi showed 3-13% increase in activity above that predicted for simple additivity in each case. Specific data are as follows:
VAL-083+Olaparib: 8.9%
VAL-083+Talazoparib: 12.6%
VAL-083+Nicaparib: 10.7%
VAL-083+Rucaparib: 11.0%
VAL-083+Veliparib: 2.9%

Each PARP inhibitor, when combined with VAL-083, produced consistent superadditivity. Of the five PARP inhibitors, olapraib, talazoparib and niraparib provided the best effects (13-24% above an additive effect). Superadditivity of the combination was also observed in a BRCA1-deficient state, with olaparib, talazoparib, niraparib and rucaparib demonstrating greater effects (9-13% above additive effects). In contrast, veliparib was not as effective. These results demonstrate that VAL-083 can synergize with PARP inhibitors in both a BRCA1-proficient and deficient state. The mechanism of action of dianhydrogalactitol, inducing DNA double-strand breaks and activating the HR repair pathway, suggests potential synergy or super-additivity with drugs that sensitize cancer cells to DNA double-strand breaks, like PARP inhibitors

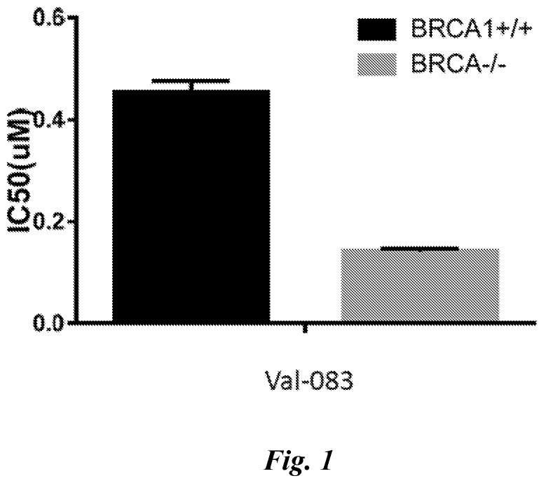

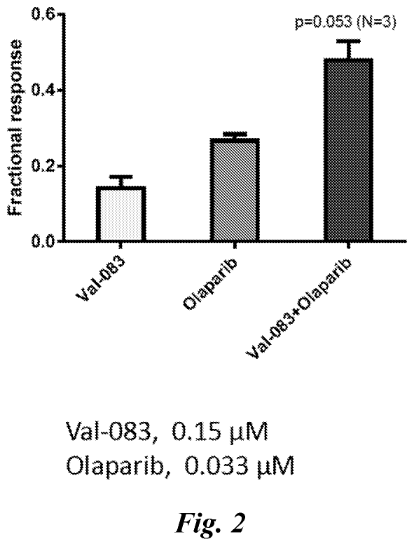

As demonstrated, for example, in FIG. 1, dianhydrogalactitol activates the HR pathway and possesses increased activity in cancers that are HR-impaired, such as BRCA-deficient or PTEN-deficient malignancies, including ovarian cancer. The potency of dianhydrogalactitol activity was increased (IC₅₀ was reduced) when HR was impaired, demonstrating that dianhydrogalactitol-induced DNA-lesions are repaired via the HR pathway. As shown in FIG. 1 and described above, dianhydrogalactitol demonstrated greater cytotoxicity (IC₅₀ was reduced) when BRCA1 was knocked down by targeting siRNA (BRCA1−/−) relative to control siRNA (BRCA1+/+). These results verify that dianhydrogalactitol-induced DNA lesions are repaired via the HR pathway, and further show that dianhydrogalactitol possesses increased activity in cancers that are HR-impaired, such as malignancies that are BRCA-deficient, or PTEN-deficient malignancies, or with other BRCAness features, including ovarian cancer. FIG. 2 shows that dianhydrogalactitol displays super-additivity with an exemplary PARP inhibitor olaparib in ovarian cancer cell line A2780 (n=3).

### Combination of Dianhydrogalactitol with Topoisomerase Inhibitors

In PC3 and A549 cells, the cytotoxic effects of dianhydrogalactitol, etoposide, and camptothecin on cell growth were determined by the crystal violet assay as described previously. The IC₅₀ values of each drug were determined using GraphPad Prism 6.0 software. For combination treatment with dianhydrogalactitol and etoposide or camptothecin, cells were exposed to fixed concentration ratios (in PC3 cells, dianhydrogalactitol:etoposide=4.55:1, dianhydrogalactitol:camptothecin=250:1; In A549 cells, dianhydrogalactitol:etoposide=5.14:1, dianhydrogalactitol:camptothecin=211.76:1) based on their corresponding IC₅₀ values for 72 h. Different combinations (e.g. ranging from one tenth of the IC₅₀ to ten times of the IC₅₀ concentrations) of each drug plus control were tested in three to four independent experiments with triplicate samples. The CI values were calculated according to the Chou-Talalay method (T. C. Chou, “Drug Combination Studies and Their Synergy Quantification Using the Chou-Talalay Method,” Cancer Res. 70: 440-446 (2010)) with Calculsyn software (Biosoft, Version 2.0) to quantitatively determine the nature of drug and drug interactions (CI<1, synergism; CI=1, additivity; CI>1, antagonism).

Table 1 shows that dianhydrogalactitol demonstrates synergy with etoposide (Type 2 topoisomerase inhibitor), and Table 2 shows that dianhydrogalactitol demonstrates synergy with camptothecin (Type 1 topoisomerase inhibitor) in PC3 prostate and A549 NSCLC cancer cells. Tables 1 and 2 show CI values for the cytotoxic effect (Fa), achieved at indicated drug combination, with CI<1 indicating synergy. Molar ratio dianhydrogalactitol:etoposide was 5:1 in PC3 and 5:1 in A549; molar ratio dianhydrogalactitol:camptothecin was 250:1 in PC3 and 212:1 in A549.

TABLE 1
Synergistic effect of VAL-083 and etoposide in PC3
and A549 cells (combination treatment for 72 h).

| Cell | Drug concentrations (μM) | | Cytotoxic | Combination |
|---|---|---|---|---|
| line | VAL-083 | Etoposide | effect (Fa) | index (CI) |
| PC3 | 4.40 | 0.97 | ED25 | 0.72 |
| | 14.47 | 3.18 | ED50 | 0.58 |
| | 48.46 | 10.66 | ED75 | 0.48 |
| | 165.40 | 36.39 | ED90 | 0.42 |
| A549 | 0.83 | 0.16 | ED25 | 0.64 |
| | 3.72 | 0.72 | ED50 | 0.72 |
| | 17.59 | 3.42 | ED75 | 0.88 |

TABLE 2
Synergistic effect of VAL-083 and camptothecin in PC3
and A549 cells (combination treatment for 72 h).

| Cell | Drug concentrations (μM) | | Cytotoxic | Combination |
|---|---|---|---|---|
| line | VAL-083 | Camptothecin | effect (Fa) | index (CI) |
| PC3 | 10.87 | 0.04 | ED40 | 0.86 |
| | 15.95 | 0.06 | ED50 | 0.80 |
| | 45.31 | 0.18 | ED75 | 0.68 |
| | 129.60 | 0.52 | ED90 | 0.59 |
| A549 | 25.26 | 0.12 | ED85 | 0.94 |
| | 41.52 | 0.19 | ED90 | 0.87 |
| | 92.81 | 0.44 | ED95 | 0.77 |

The tables show CI values for the cytotoxic level (Fa) shown, achieved at indicated drug concentrations. CI<1 shows synergy. Mean+/−SE, N=4-7.

### Combinations of Dianhydrogalactitol with Other p53 Modulators

To further explore potential therapeutic combinations with VAL-083, other targeted drugs were also evaluated. The drugs used were supplied by DelMar (VAL-083) or obtained from Selleckchem (BKM120, GSK2830371, MK-1775, Nutlin-3a, and palbociclib), and Dr. James Bradner as a gift (JQ1). Stock solutions were prepared in water (VAL-083 and palbociclib) or DMSO (all others) and frozen at −20° C. The drug was added to complete RPMI 1640 media containing 10% fetal bovine serum for a final working stock solution at a desirable drug concentration that was used either immediately or after a 24-hr incubation at 37° C. to assess biological activity against A2780 cells by standard 5-day MTT cytotoxic assay (Carmichael J, DeGraff W G, Gazdar A F, Minna J D, Mitchell J B. Evaluation of a tetrazolium-based semiautomated colorimetric assay: assessment of chemosensitivity testing. Cancer Res. 1987; 47:936-942). The IC₅₀ values (concentration inhibiting growth of cells by 50%) were generated by fitting Fa values from a range of drug concentrations to a four-parameter logistic dose-response sigmoidal equation using the Graphpad Prism v.6 software.

Combination studies also utilized the 5-day MTT assay, and the fraction of cells affected (Fa) at a specified concentration of drug A alone, drug B alone or combination of drug A+drug B. Superadditivity was assessed from the determination of predicted additive effect of [drug A+drug B] combination, based on the equation described by Tallarida et. al (Tallarida R J. Drug synergism: its detection and applications. J Pharmacol Exp Ther. 2001; 298:865-872), as follows:

Additive effect of [drug A+drug B]=FaA+(1−FaA)FaB

where FaA or FaB=fraction of cells affected (Fa) by drug A or B alone. Data, where appropriate, are provided as Mean±SE of N=3 or greater.

Positive drug combinations were evaluated for possible underlying mechanism 24 hr after drug exposures using protein-specific antibodies to develop immunoblots. The methodologies for these studies are described in detail in publications (Kuang J, He G, Huang Z, Khokhar A R, Siddik Z H. Bimodal effects of 1R,2R-diaminocyclohexane(trans-diacetato)(dichloro)platinum(IV) on cell cycle checkpoints. Clin Cancer Res. 2001; 7:3629-3639. He G, Kuang J, Khokhar A R, Siddik Z H. The impact of S- and G2-checkpoint response on the fidelity of G1-arrest by cisplatin and its comparison to a non-cross-resistant platinum(IV) analog. Gynecol Oncol. 2011; 122:402-409. He G, Kuang J, Koomen J, Kobayashi R, Khokhar A R, Siddik Z H. Recruitment of trimeric proliferating cell nuclear antigen by G1-phase cyclin-dependent kinases following DNA damage with platinum-based antitumour agents. Br J Cancer. 2013; 109:2378-2388).

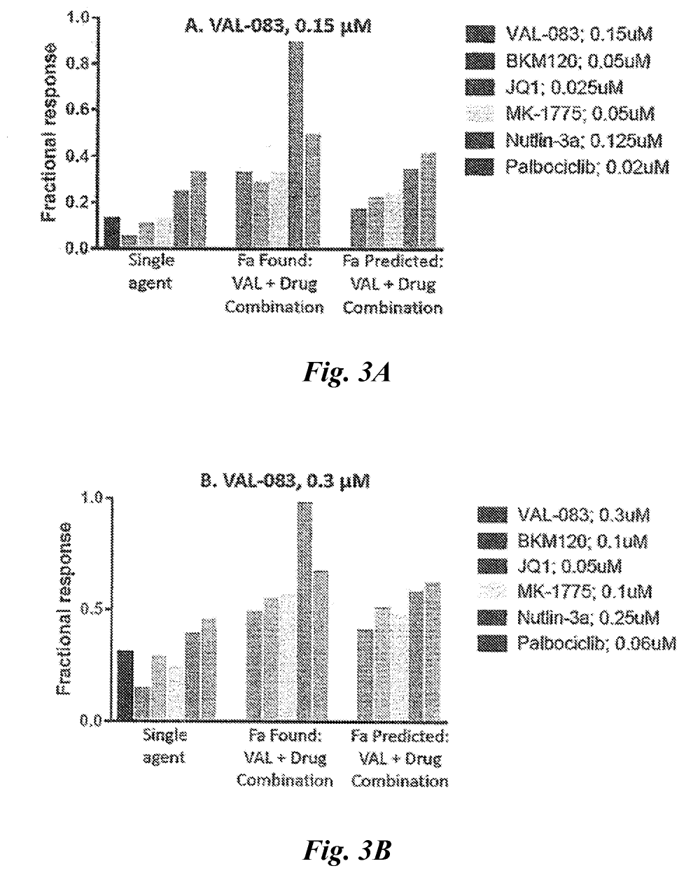

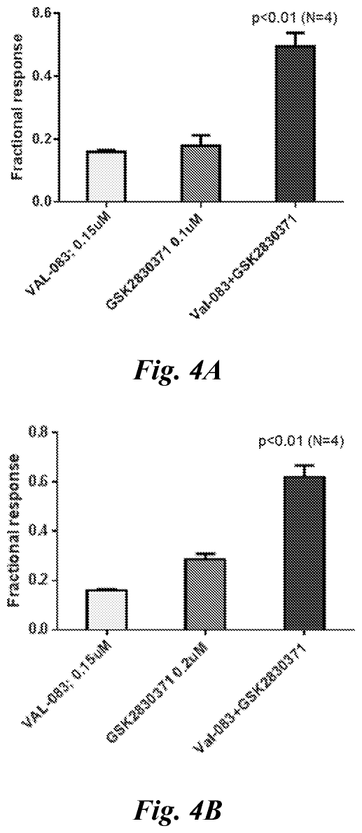

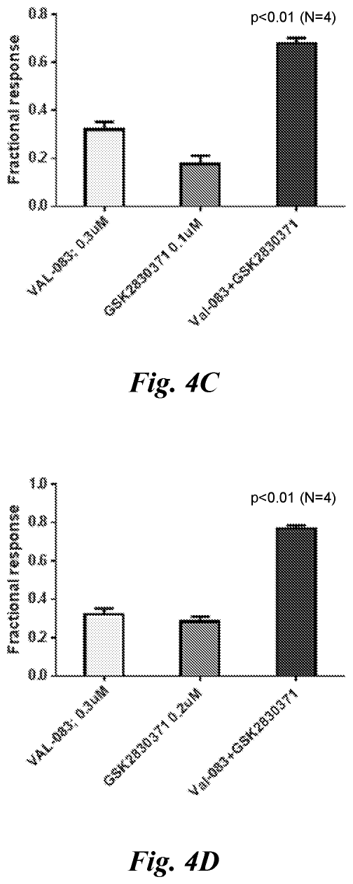

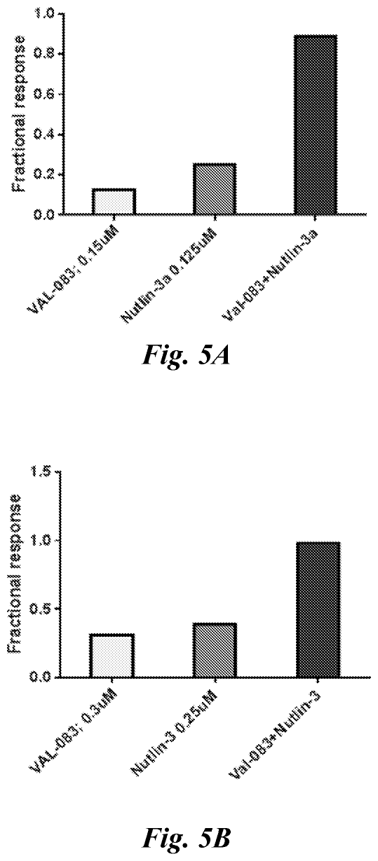

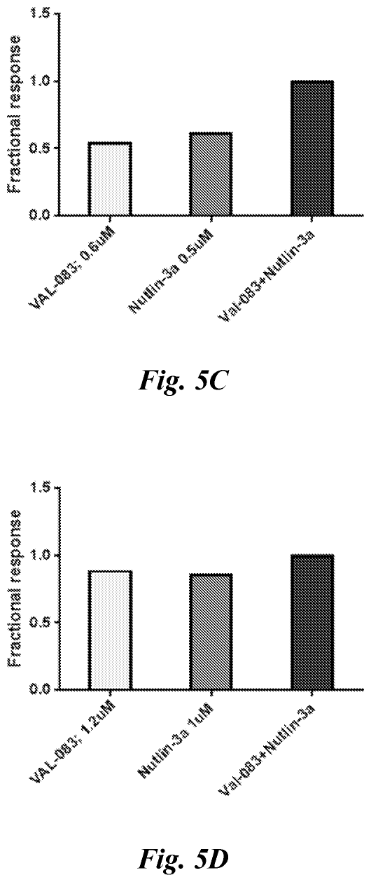

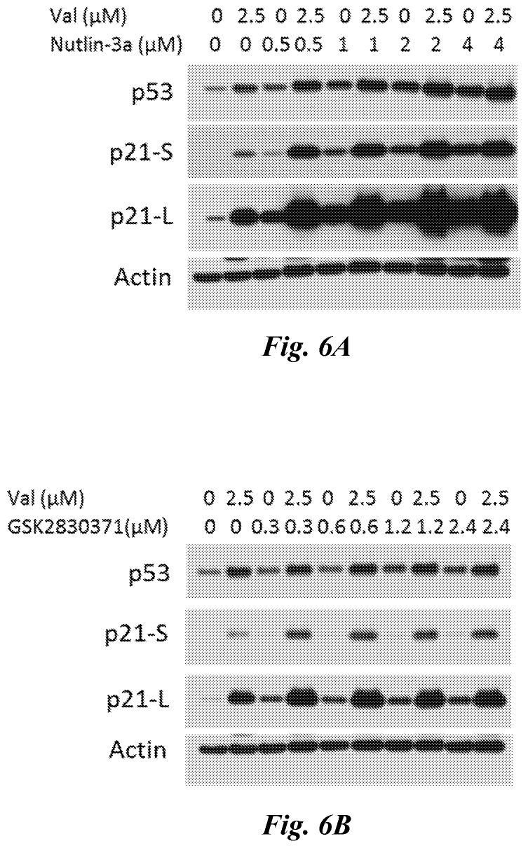

As shown in FIG. 3, dianhydrogalactitol cytotoxicity was augmented by non-DNA targeting agents (p53 modulators) as determined by the MTT assay using the standard 5-day drug exposure protocol. Although BKM120, JQ1, MK1775 and palbociclib did not result in superadditive activity, the p53 modulators GSK2830371 or nutlin-3a resulted in substantial superadditivity and/or synergy in combination with VAL-083. Binary combinations of VAL-083 in A2780 cells were most effective with GSK2830371 and nutlin-3a, which are inhibitors that upregulate p53 function by inhibiting dephosphorylation of p53 or its binding to MDM2, respectively.

The p53 activator nutlin-3a does not, as a single agent, induce DNA damage, but nutlin-3a augmented the activity of dianhydrogalactitol in ovarian cancer cell line A2780 cells, which have wild-type p53. The superadditiviy of nutlin-3a was further examined by assessment of combination index, which was in the range 0.10-0.15 and indicative of strong synergy, as shown in Table 3.

TABLE 3
Combination of VAL-083 (DAG) with Nutlin-3a demonstrates
synergistic effect in A2780 cells.

| | |
|---|---|
| Cytotoxic | Concentration (μM) |

| Level (Fa) | VAL-083 | Nutlin-3a | CI |
|---|---|---|---|
| ED75 | 0.84 | 0.7 | 0.10 |
| ED90 | 1.52 | 1.6 | 0.13 |
| ED95 | 2.3 | 2.76 | 0.15 |

The table shows CI values for the cytotoxic level (Fa) shown. CI<1 shows synergy

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including issued patents, published patent publications, and journal articles, are incorporated herein by this reference.

While illustrative embodiments have been illustrated and described, it will be appreciated that various changes can be made therein without departing from the spirit and scope of the invention.

The embodiments of the invention in which an exclusive property or privilege is claimed are defined as follows:

1. A composition for treating a malignancy comprising an alkylating hexitol, a p53 modulator, and optionally one or more pharmaceutically acceptable carriers.

2. The composition of claim 1, wherein the p53 modulator is nutlin-3a or GSK2830371.

3. The composition of claim 1, wherein the alkylating hexitol derivative is dianhydrogalactitol, diacetyldianhydrogalactitol, or dibromodulcitol.

4. The composition of claim 3, wherein the alkylating hexitol derivative is dianhydrogalactitol.

5. The composition of claim 1, wherein the alkylating hexitol is included in a therapeutically effective quantity.

6. The composition of claim 1, wherein p53 modulator is included in a therapeutically effective quantity.

7. The composition of claim 6, wherein the therapeutically effective quantities of the alkylating hexitol derivative and the p53 modulator are quantities that produce synergism between the activities of the alkylating hexitol derivative and the p53 modulator.

8. The composition of claim 1, wherein the alkylating hexitol derivative and the p53 modulator are included in separate containers, each of which optionally comprises a pharmaceutically acceptable carrier.

9. The composition of claim 8, wherein the composition comprises a treatment regimen.

10. A method for treating a malignancy comprising the steps of:
(a) administering a therapeutically effective quantity of an alkylating hexitol derivative; and
(b) administering a therapeutically effective quantity of a p 53 modulator;
in order to treat the malignancy.

11. The method of claim 10, wherein the alkylating hexitol derivative is dianhydrogalactitol, diacetyldianhydrogalactitol, or dibromodulcitol.

12. The method of claim 11, wherein the alkylating hexitol derivative is dianhydrogalactitol.

13. The method of claim 10, wherein the p53 modulator is nutlin-3a or GSK2830371.

14. The method of claim 10, wherein the malignancy is ovarian cancer.

15. The method of claim 14, wherein the ovarian cancer is platinum-resistant ovarian cancer.

16. The method of claim 10, wherein the malignancy is BRCA-deficient ovarian cancer.

17. The method of claim 10, wherein the malignancy is BRCAness ovarian cancer.

18. The method of claim 10, wherein the malignancy is a high-grade serous ovarian carcinoma.

19. The method of claim 10, wherein the malignancy is BRCA-proficient ovarian cancer.

20. The method of claim 10, wherein the steps are performed sequentially in any order or simultaneously.

21. A composition for treating a malignancy comprising a therapeutically effective quantity of an alkylating hexitol, a therapeutically effective quantity of a PARP inhibitor, and optionally one or more pharmaceutically acceptable carriers.

22. The composition of claim 21, wherein the alkylating hexitol derivative is dianhydrogalactitol, diacetyldianhydrogalactitol, or dibromodulcitol.

23. The composition of claim 22, wherein the alkylating hexitol derivative is dianhydrogalactitol.

24. The composition of claim 21, wherein the PARP inhibitor is olaparib, talazoparib, niraparib, or rucaparib.

25. The composition of claim 24, wherein the PARP inhibitor is olaparib.

26. The composition of claim 21, wherein the alkylating hexitol derivative is dianhydrogalactitol and the PARP inhibitor is olaparib.

27. The composition of claim 21, wherein the alkylating hexitol derivative and the PARP inhibitor are in separate pharmaceutical carriers or in the same pharmaceutical carrier.

28. The composition of claim 21, wherein the therapeutically effective quantities of the alkylating hexitol derivative and the PARP inhibitor are quantities that produce synergism between the activities of the alkylating hexitol derivative and the PARP inhibitor.

29. A method for treating a malignancy comprising the steps of:
(a) administering a therapeutically effective quantity of an alkylating hexitol derivative; and
(b) administering a therapeutically effective quantity of a PARP inhibitor;
in order to treat the malignancy.

30. The method of claim 29, wherein the malignancy is ovarian cancer.

31. The method of claim 30, wherein the ovarian cancer is platinum-resistant ovarian cancer.

32. The method of claim 29, wherein the malignancy is BRCA-deficient ovarian cancer.

33. The method of claim 29, wherein the malignancy is BRCAness ovarian cancer.

34. The method of claim 29, wherein the malignancy is high-grade serous ovarian carcinoma.

35. The method of claim 29, wherein the malignancy is BRCA-proficient ovarian cancer.

36. The method of claim 29, wherein the alkylating hexitol derivative is dianhydrogalactitol, diacetyldianhydrogalactitol, or dibromodulcitol.

37. The method of claim 29, wherein the alkylating hexitol derivative is dianhydrogalactitol.

38. The method of claim 29, wherein the PARP inhibitor is olaparib, talazoparib, niraparib or rucaparib.

39. The method of claim 29, wherein the PARP inhibitor is olaparib.

40. The method of claim 29, wherein the alkylating hexitol derivative is dianhydrogalactitol and the PARP inhibitor is olaparib.

41. The method of claim 29, wherein the steps are performed sequentially in any order or simultaneously.

42. A method for treating a malignancy comprising the steps of:
(a) administering a therapeutically effective quantity of an alkylating hexitol derivative; and
(b) administering a therapeutically effective quantity of a topoisomerase inhibitor;
in order to treat the malignancy.

43. The method of claim 42, wherein the alkylating hexitol derivative is dianhydrogalactitol, diacetyldianhydrogalactitol, or dibromodulcitol.

44. The method of claim 43, wherein the alkylating hexitol derivative is dianhydrogalactitol.

45. The method of claim 42, wherein the topoisomerase inhibitor is a Type 1 topoisomerase inhibitor.

46. The method of claim 42, wherein the topoisomerase inhibitor is a Type 2 topoisomerase inhibitor.

47. The method of claim 42, wherein the topoisomerase inhibitor is a Type 1/type 2 topoisomerase inhibitor.

48. The method of claim 42, wherein the topoisomerase inhibitor is camptothecin, irinotecan, doxorubicin, topotecan, etoposide, or mitoxantrone.

49. The method of claim 42, wherein the topoisomerase inhibitor is camptothecin.

50. The method of claim 42, wherein the topoisomerase inhibitor is etoposide.

51. The method of claim 42, wherein the malignancy is non-small cell lung cancer (NSCLC).

52. The method of claim 51, wherein the NSCLC is cisplatin-resistant NSCLC.

53. The method of claim 42, wherein the malignancy is prostate cancer.

54. The method of claim 42, wherein the alkylating hexitol derivative is dianhydrogalactitol and the topoisomerase inhibitor is camptothecin.

55. The method of claim 42, wherein the alkylating hexitol derivative is dianhydrogalactitol and the topoisomerase inhibitor is etoposide.

56. The method of claim 42, wherein the steps are performed sequentially in any order or simultaneously.

57. The method of claim 42, wherein the therapeutically effective quantities of the alkylating hexitol derivative and the topoisomerase inhibitor are quantities that produce synergism between the activities of the alkylating hexitol derivative and the topoisomerase inhibitor.
